(19) United States
(12) Patent Application Publication
(10) Pub. No.: US 2024/0029014 A1
(43) Pub. Date: Jan. 25, 2024

(54) ELECTRONIC METHODS AND SYSTEMS FOR FACILITATING CONSUMER-CENTRIC MANUFACTURING OF SUPPLEMENTS

(71) Applicant: NONSTOP EVOLVING CORP., La Jolla, CA (US)

(72) Inventor: Sami CHOURA, La Jolla, CA (US)

(21) Appl. No.: 17/708,880

(22) Filed: Mar. 30, 2022

(51) Int. Cl. G06Q 10/08; G06Q 30/06; G16H 20/10; G16H 10/20

(52) U.S. Cl. CPC G06Q 10/087; G06Q 30/0621; G16H 20/10; G16H 10/20

(57) ABSTRACT

Embodiments of the present disclosure provide methods and systems for facilitating manufacturing of the supplements unique to each user. The method performed by a server system includes receiving a request to manufacture a supplement through a health application available on a user device associated with a user. The method includes receiving a user survey response from the user. The method includes extracting user information from a plurality of sources, external databases and the user survey response. The method includes determining one or more micronutrients of the supplement and one or more parameters associated with each micronutrient based on the user information and artificial intelligence (AI) model. The one or more parameters include at least dosage value, serving form, and serving frequency of the supplement. Further, the method includes facilitating manufacturing of the supplement for the user in a timely manner based on monitoring a digital inventory of the user.

### TECHNICAL FIELD

The present disclosure relates to server-based electronic methods and systems for manufacturing supplements based on the specific needs of the users or consumers.

### BACKGROUND

Most people try to manage health and wellness through a combination of exercise and a balanced diet. However, quite often, people are unable to pay attention to their diet, consume junk food, or skip meals that result in unbalanced diets (or insufficiency of essential nutrients like vitamins and minerals in the body). To overcome these, various systems and methods are implemented by supplement manufacturers to manufacture supplements containing essential nutrients and micronutrients for the users.

Currently, the systems and methods implemented by the supplement manufacturers employ a generic manufacturing technique for manufacturing the supplements. The supplements manufactured using the generic manufacturing process contain a predefined or fixed amount of ingredients (i.e. the essential nutrients and micronutrients). However, intake of such supplements may cause the users to overdose on ingredients as the requirement of the dietary supplements in the body changes with time. Further, the quantity or dosage value of the supplements differs for each individual based on his/her lifestyle. Additionally, the supplements are made available in the market based on the results obtained from conventional artificial testing procedures (testing the supplements on beta test users (e.g. cells or animals) under controlled environments). However, the results obtained from the conventional artificial test procedures can be inaccurate. Thus, intake of such supplements may not benefit the users and in fact, can lead to various health issues.

Moreover, due to a busy schedule, it may be difficult for the users to track the quantity of the supplements, thus leaving gaps in their daily consumption needs. To overcome such conditions, the users may opt for a subscription with a corresponding supplement manufacturer for auto-delivering the supplements periodically (e.g., monthly basis). However, in some instances, the user may receive the next shipment of the supplements while the supplements delivered in the previous shipment are still available with the user. Thus, the subscription for auto-delivering the supplements to the user is unregulated and uneconomical for the user.

Therefore, there is a need for a technique that can overcome one or more limitations stated above in addition to providing other technical advantages.

### SUMMARY

Various embodiments of the present disclosure provide electronic methods and systems for facilitating consumer-centric manufacturing of the supplements that is unique to each consumer.

In an embodiment, a computer-implemented method is disclosed. The computer-implemented method performed by a server system includes receiving a request to manufacture a supplement through a health application available on a user device associated with a user. The computer-implemented method includes receiving a user survey response from the user through the health application. The user survey response includes data related to health profile of the user at the time of requesting to manufacture the supplement. The computer-implemented method further includes extracting user information from at least a plurality of sources, external databases, and the user survey response. The computer-implemented method includes determining one or more micronutrients of the supplement and one or more parameters associated with each micronutrient of the one or more micronutrients based at least on the user information and artificial intelligence (AI) model. The one or more parameters associated with each of the one or more micronutrients for manufacturing the supplement for the user include at least an optimum dosage value, an optimal serving form, and an optimal serving frequency of the supplement. Further, the computer-implemented method includes facilitating manufacturing of the supplement for the user in a timely manner based at least on monitoring a digital inventory associated with the user in the health application.

In another embodiment, a server system is disclosed. The server system includes a communication interface, a memory including executable instructions, and a processor communicably coupled to the communication interface and the memory. The processor is configured to cause the server system to perform at least in part to receive a request to manufacture a supplement through a health application available on a user device associated with a user. The server system is caused to receive a user survey response from the user through the health application. The user survey response includes data related to health profile of the user at the time of requesting to manufacture the supplement. The server system is further caused to extract user information from at least a plurality of sources, external databases, and the user survey response. The server system is caused to determine one or more micronutrients of the supplement and one or more parameters associated with each micronutrient of the one or more micronutrients based at least on the user information and artificial intelligence (AI) model. The one or more parameters associated with each of the one or more micronutrients for manufacturing the supplement for the user includes at least an optimum dosage value, an optimal serving form, and an optimal serving frequency of the supplement. Further, the server system is caused to facilitate manufacturing of the supplement for the user in a timely manner based at least on monitoring a digital inventory associated with the user in the health application.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device, or a tool and instrumentalities disclosed herein. Moreover, those in the art will understand that the drawings are not to scale.

FIG. 1 illustrates an exemplary representation of an environment related to at least some embodiments of the present disclosure;

FIG. 2 represents a sequence flow diagram for facilitating Supplement-Manufacturing-as-a-service through health application to a user, in accordance with an embodiment of the present disclosure;

FIG. 3 represents a process flow for optimizing subsequent manufacturing of the supplement for the user via a feedback loop facilitated through the health application, in accordance with an embodiment of the present disclosure;

FIG. 4 illustrates an example representation of a user interface (UI), depicting a request to manufacture the supplement initiated by the user to a server system through the health application, in accordance with an embodiment of the present disclosure;

FIG. 5 illustrates an example representation of a user interface, depicting a digital inventory associated with the user in the health application, in accordance with an embodiment of the present disclosure;

FIG. 6 illustrates an example representation of a user interface, depicting a feedback form rendered on the health application, in accordance with an embodiment of the present disclosure;

FIG. 7 represents an example scenario depicting a comparison result of two or more supplements rendered on an extended reality (XR) device associated with the user, in accordance with an embodiment of the present disclosure;

FIG. 8 illustrates a flow diagram of a computer-implemented method for facilitating dynamic manufacturing of the supplement for the user, in accordance with an embodiment of the present disclosure;

FIG. 9 is a simplified block diagram of a server system for facilitating dynamic manufacturing of the supplement for the user, in accordance with one embodiment of the present disclosure; and

FIG. 10 is a simplified block diagram of an electronic device capable of implementing various embodiments of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

### Overview

Various examples of the present disclosure provide electronic methods and systems for facilitating dynamic manufacturing of the supplements unique to each consumer (or user).

The present disclosure discloses a server system configured to facilitate manufacturing of a supplement directly and unique to the user based on considering user profile and various laboratory test reports. The server system is configured to receive a request to manufacture the supplement through a health application available on a user device associated with the user. The request includes information related to supplement name, quantity, micronutrients, and the like. The user submits the request to manufacture the supplement when the supplement is nearing full consumption. Upon receipt of the request, the server system transmits a user survey to the user. As a result, the server system receives a user survey response from the user through the health application. The user survey response includes data related to a health profile of the user at the time of requesting to manufacture the supplement. The user survey including user inputs corresponds to the user survey response. Thereafter, the server system extracts user information from at least a plurality of sources, external databases, and the user survey response. The user information includes data related to a plurality of supplements included in the digital inventory, health metrics, dosage values, serving form, and quantity of each micronutrient available in the user's body. The server system further determines one or more micronutrients of the supplement and one or more parameters associated with each micronutrient based on the user information and artificial intelligence (AI) model. The one or more parameters associated with each of the one or more micronutrients include at least an optimum dosage value, an optimal serving form, and an optimal serving frequency of the supplement. In addition, the server system determines a time schedule to manufacture and deliver the supplement to the user based on the digital inventory associated with the user. The supplement data including the one or more micronutrients of the supplement, the one or more parameters associated with each micronutrient, and the time schedule is transmitted to a manufacturing application available on a user device associated with a supplement manufacturer.

In addition, the server system rewards the user with reward points in form of digital currency based at least on the consumption habits of the user, purchasing supplements in the health application, and user activities in the health application. For example, the reward points in the form of digital currency provided to the user are crypto-collectibles such as non-fungible tokens (NFTs).

Further, the server system receives a feedback response from the user through the health application, following the consumption of the supplement manufactured by the user. The server system determines the efficacy of the supplement manufactured for the user based, at least in part, on the feedback response. Additionally, the server system provides recommendations to optimize subsequent manufacturing of the supplement for the user, if the efficacy of the supplement on the user is less than a predefined value. The recommendations for optimizing subsequent manufacturing of the supplement include information related to optimum dosage values and serving form to be selected for each micronutrient of the supplement.

The server system facilitates the display of a comparison result of two or more supplements on an extended reality (XR) device associated with the user. The comparison result provides a comparison of the two or more supplements on an ingredient level including a list of micronutrients associated with each of the two or more supplements. The user can utilize the comparison result to purchase a suitable supplement from the two or more supplements available in the health application. Moreover, the server system is configured to facilitate the display of an alert message in form of an indicator corresponding to at least one micronutrient of the list of micronutrients based on determining that at least one micronutrient is incompatible for the user.

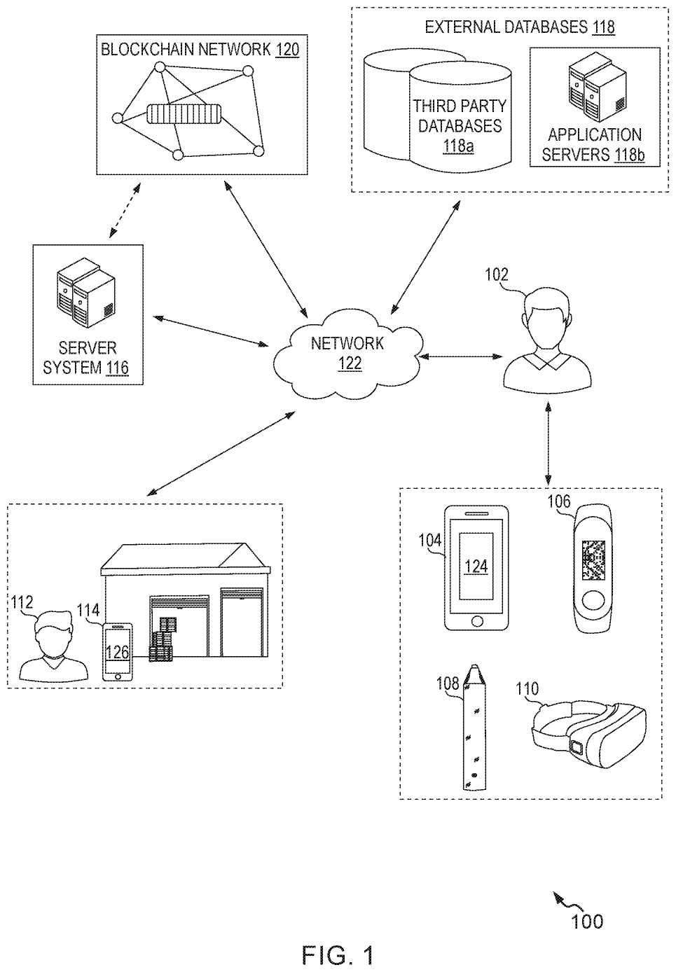

Various example embodiments of the present disclosure are described hereinafter with reference to FIG. 1 to FIG. 10.

FIG. 1 illustrates an exemplary representation of an environment 100 related to at least some example embodiments of the present disclosure. Although the environment 100 is presented in one arrangement, other embodiments may include the parts of the environment 100 (or other parts) arranged otherwise depending on, for example, manufacturing supplements uniquely and directly to users based on considering user profile and micronutrients requirements of each user. The environment 100 includes a user 102 (i.e. consumer) associated with a user device 104 (exemplary depicted to be a “mobile phone”), one or more wearable devices, and nutrient trackers (such as a wearable device 106 and a nutrient tracker 108), and an extended reality (XR) device 110. The wearable device 106, the nutrient tracker 108, and the XR device 110 can be connected with the user device 104 via a short-range communication protocol such as, but not limited to, Bluetooth, ZigBee, Z-wave, and the like. Further, the environment 100 includes a supplement manufacturer 112 associated with a user device 114, a server system 116, external databases 118, and a blockchain network 120. The external databases 118 include one or more third-party databases 118a and one or more application servers 118b associated with one or more applications used by the user 102.

Various entities in the environment 100 (as shown in FIG. 1) may connect to a network 122 in accordance with various wired and wireless communication protocols, such as Transmission Control Protocol and Internet Protocol (TCP/IP), User Datagram Protocol (UDP), 2nd Generation (2G), 3rd Generation (3G), 4th Generation (4G), 5th Generation (5G) communication protocols, Long Term Evolution (LTE) communication protocols, future communication protocols, or any combination thereof. For example, the network 122 may include, without limitation, multiple different networks, such as a private network made accessible by the server system 116, separately, and a public network (e.g., the Internet). Further, the network 122 may include, a light fidelity (Li-Fi) network, a local area network (LAN), a wide area network (WAN), a metropolitan area network (MAN), a satellite network, the Internet, a fiber-optic network, a coaxial cable network, an infrared (IR) network, a radio frequency (RF) network, a virtual network, and/or another suitable public and/or private network capable of supporting communication among the entities illustrated in FIG. 1, or any combination thereof.

The environment 100 including the server system 116 is configured to perform one or more of the operations described herein. In general, the server system 116 is configured to facilitate dynamic manufacturing of the supplements to the user (such as the user 102) based on extracting actual data related to the health profile and requirements of the user 102. The server system 116 is a separate part of the environment 100 and may operate apart from (but still in communication with, for example, via the network 122) any third party external servers (to access data to perform the various operations described herein).

The server system 116 provides an instance of a health application 124 to the user device 104 of the user 102 for monitoring the micronutrients requirements of the user 102. The components and/or features associated with the health application 124 may rest in the server system 116 and the user device 104. In general, the health application 124 managed by the server system 116 performs one or more operations such as, but not limited to, monitoring micronutrients intake, supplements stock, and the health profile of the user 102. Further, the health application 124 is configured to receive information from the external databases 118 and a plurality of sources on a periodic basis. The plurality of sources constitutes the devices and gadgets (such as the user device 104, the nutrient tracker 108, and the wearable device 106) associated with the user 102 and a user profile of the user 102 maintained in the health application 124.

More specifically, the wearable device 106 may provide information related to various parameters associated with the user 102 such as, but not limited to, sleep activity, exercise time, calorie burnt, pulse rate, and the like. The nutrient tracker 108 may provide information related to nutrients intake of the user 102, supplement consumption activities, amount of each nutrient in the user's body over a period of time, and the like. Further, the third-party databases 118a may include information associated with the user 102 related to one or more laboratory test reports (e.g., body/blood tests, health activities, medical consultation details, etc.). Furthermore, the health application 124 may access one or more applications (managed by the application servers 118b) available on the user device 104 for extracting additional information pertaining to the user 102. The applications used by the user 102 that are managed by corresponding application servers (i.e. the application servers 118b) may include applications associated with the wearable device 106 and the nutrient tracker 108, a fitness application, a sleep monitoring application, and the like. The server system 116 managing the health application 124 recommends supplements, their dosage values, serving form, and serving frequency, and a nutrition goal to the user 102 based on the information extracted from the plurality of sources and the external databases 118.

Further, the server system 116 offers a service such as Supplements-Manufacturing-as-a-service through the health application 124 to the user 102. More specifically, the server system 116 is configured to receive a request to manufacture a supplement through the health application 124 available on the user device 104 of the user 102. Upon receipt of the request, the server system 116 transmits a user survey to the user 102 for receiving inputs related to the health profile of the user 102 at the time of requesting to manufacture the supplement. Further, the server system 116 extracts data pertaining to the user 102 from at least the plurality of sources (i.e. the user profile of the user 102, the user device 104, the nutrient tracker 108, and the wearable device 106) and the laboratory results from the external databases 118. The data extracted from the plurality of sources, user survey response, and the external databases 118 constitute user information of the user 102.

Thereafter, the server system 116 determines one or more micronutrients of the supplement and one or more parameters associated with each micronutrient based at least on the user information and artificial intelligence (AI) model. The AI model associated with the server system 116 determines the micronutrients requirements of the user 102 based on the user information and facilitates manufacturing of the supplement requested by the user 102 in an optimized, homogeneous and timely manner to the user 102.

The term “artificial intelligence (AI) model”, used throughout the description, may refer to an application of artificial intelligence that provides systems with the ability to automatically learn and improve from experience without explicitly being programmed. An artificial intelligence model may include a set of software routines and parameters that can predict an output of a process (e.g., the micronutrients requirement of the user for facilitating manufacturing of the supplement in an optimized manner, etc.) based on a “feature vector” or other input data (i.e. the user information). A structure of the software routines (e.g., number of subroutines and the relation between them) and/or the values of the parameters can be determined in a training process, which can use actual results of the process that is being modeled. Examples of artificial intelligence models include machine learning models, such as support vector machines (SVMs), models that classify data by establishing a gap or boundary between inputs of different classifications, as well as neural networks, which are collections of artificial “neurons” that perform functions by activating in response to inputs. In some embodiments, a neural network can include a convolutional neural network (CNN), a recurrent neural network (RNN), etc.

The server system 116 determines a time schedule to manufacture and ship the supplement to the user 102. Thereafter, the server system 116 identifies a supplement manufacturer (e.g., the supplement manufacturer 112) to manufacture the supplement for the user 102 based on the recommendations provided by the AI model related to the micronutrients and their optimum dosages. The supplement manufacturer 112 may be equipped with necessary manufacturing equipment and raw materials for manufacturing the supplement for the user 102 as indicated by the server system 116. Thus, the server system 116 ensures manufacturing and delivery of the supplement to the user 102 in a timely manner. As a result, the gaps in the consumption habits of the user 102 are prevented and it allows proper management of the micronutrients associated with the user 102.

Further, the user 102 is rewarded with reward points in form of digital currency based at least on consumption habits (or supplements intake) of the user 102, purchasing supplements in the health application 124, and user activities (e.g., attending health events) in the health application 124. In one example, the digital currency is crypto collectibles (e.g., non-Fungible tokens (NFT)) that are stored in the blockchain network 120 and made accessible to the server system 116 for rewarding the user 102. The user 102 can redeem the reward points (i.e. digital currency) in the health application 124 for purchasing the supplements offered by the health application 124. In an embodiment, the user 102 can redeem the reward points (i.e. the crypto convertibles) at other vendors or items or events that are associated with the health application 124 and/or the server system 116. This provides additional benefits to the user 102 which allows the user 102 to engage with other supplements offered by the vendors, participate in health-related events, etc.

Additionally, the server system 116 is configured to employ a smart formulation technique to optimize subsequent manufacturing of the supplement for the user 102 based at least on receipt of a feedback response from the user 102. In general, the feedback response may include information related to the health profile of the user 102 post consumption of the supplement manufactured for the user 102.

In an embodiment, the server system 116 is configured to provide a set of features in the health application 124 for establishing a connection between the XR device 110 of the user 102 and the health application 124. The user 102 can use the XR device 110 for scanning and/or capturing a nutrition label of any supplement for importing data of the nutrition label to the health application 124. Further, the server system 116 may render a comparison of two or more supplements on an ingredient level including a list of micronutrients on the XR device 110 of the user 102. The user 102 may purchase a suitable supplement amongst the two or more supplements based on the comparison. In addition, the server system 116 determines if at least one micronutrient from the list of micronutrients is incompatible with the user 102. To that effect, the server system 116 displays an alert message in the form of an indicator corresponding to the at least one micronutrient in the health application 124. Furthermore, the user 102 can customize a digital inventory associated with the user 102 in the health application 124 by providing inputs on a list of customizable settings of the health application 124.

The number and arrangement of systems, devices, and/or networks shown in FIG. 1 are provided as an example. There may be additional systems, devices, and/or networks; fewer systems, devices, and/or networks; different systems, devices, and/or networks; and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices. Additionally or alternatively, a set of systems (e.g., one or more systems) or a set of devices (e.g., one or more devices) of the environment 100 may perform one or more functions described as being performed by another set of systems or another set of devices of the environment 100.

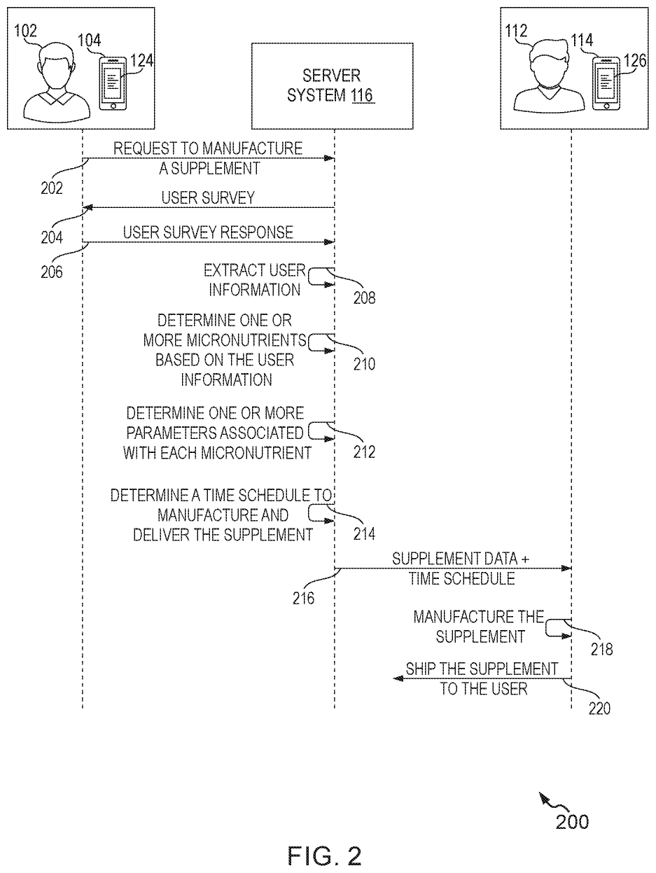

FIG. 2 represents a sequence flow diagram 200 for facilitating Supplements-Manufacturing-as-a-service through the health application 124 to a consumer (such as the user 102), in accordance with an embodiment of the present disclosure. Further, the steps of the sequence flow diagram 200 may not be necessarily executed in the same order as they are presented. Further, one or more steps may be grouped and performed in form of a single step, or one step may have several sub-steps that may be performed in parallel or a sequential manner.

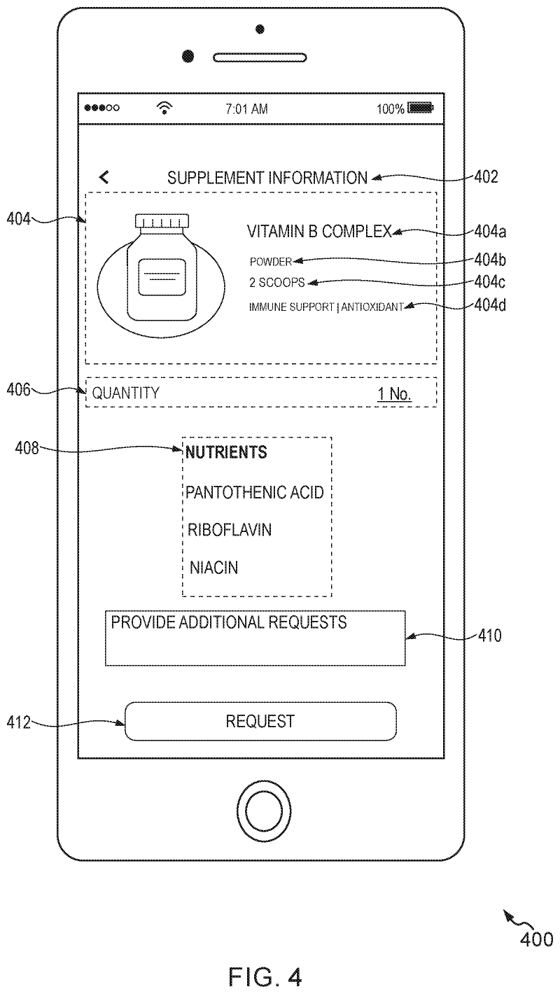

At step, 202 the user 102 transmits a request to manufacture a supplement to the server system 116 through the health application 124. In an example, the supplement requested by the user 102 may be a supplement among a plurality of supplements owned by the user 102 in the digital inventory of the health application 124. The user 102 may determine an amount of the supplement that is possessed by the user 102 is nearing full consumption. In this scenario, the user 102 initiates the request to the server system 116 by providing inputs related to the supplement in the health application 124. The request includes information related to the supplement such as, but not limited to, supplement name, micronutrients associated with the supplement, supplement quantity, and the like which is explained with reference to FIG. 4.

At step 204, the server system 116 renders a user survey in the health application 124 for receiving inputs related to the health profile of the user 102 at the time of requesting to manufacture the supplement. In general, the user's health profile changes with time. As such, variables relating to changes in the user's health profile are listed in the user survey and transmitted to the user 102. For example, the user survey may include the variables such as, but not limited to, health goals (e.g., mental focus, hair & skin, DNA repair, targeting a specific issue, etc.), user preferences (e.g., vegan or non-vegetarian), user's present condition (e.g., pregnant, lactating, allergy, etc.), details pertaining to other medications consumed by the user, and the like. At step 206, the user 102 transmits a user survey response to the server system 116 through the health application 124 upon providing user inputs for each of the variables listed in the user survey. In other words, the user survey including the user inputs corresponds to the user survey response. The user survey response determines a unique condition of the user 102 at the time of requesting to manufacture the supplement.

At step 208, the server system 116 extracts user information pertaining to the user 102 from at least the plurality of sources, the user survey response, and the external databases 118. It is to be noted that the server system 116 extracts the user information pertaining to the user 102 from at least the plurality of sources and the external databases 118 on a periodic basis (e.g., weekly, monthly, etc.). The server system 116 accesses the user profile of the user 102 in the health application 124 to determine health metrics of the user 102, the plurality of supplements owned in the digital inventory of the user 102 and their dosage values, serving form, a quantity of each micronutrient available in the user's body and the like. The health metrics may include, but are not limited to, age, body mass index (BMI), and activity level (e.g., sedentary, moderate, active, etc.). As explained above, the nutrient tracker 108, the wearable device 106, and other applications available on the user device 104 track and/or monitor parameters relating to the health and wellness of the user 102. As such, the health application 124 managed by the server system 116 accesses the information determined by the nutrient tracker 108, the wearable device 106, and other applications available on the user device 104. Thereafter, the server system 116 determines the quantity of each micronutrient available in the user's body. Further, the server system 116 may update the health metrics of the user 102 on a periodic basis based on the information from the user profile, the nutrient tracker 108, the wearable device 106, and other applications available on the user device 104.

In an embodiment, the user 102 may not own the gadgets such as the nutrient tracker 108 and the wearable device 106. In this scenario, the server system 116 extracts the data pertaining to the nutrient tracker 108 and the wearable device 106 for the user 102 from the pool of data pertaining to other users/consumers available in the database associated with the server system 116. More specifically, the server system 116 is configured to compare the user profile of the user 102 (or the consumer) with the user profiles of the other users of the health application 124. Thereafter, the server system 116 may compute a similarity score based on comparing the user profile of the user 102 with each user profile of the other users of the health application 124 for determining the match for the user profile of the user 102. The server system 116 extracts for the user 102 from the user profile of other users that is determined to be a match for the user profile of the user 102. More specifically, the server system 116 determines the information that is detected by the nutrient tracker 108 and the wearable device 106 for the user 102 based on the user profile of the other user that is determined to be the match for the user 102.

In addition, the server system 116 accesses the external databases 118 to retrieve the laboratory test reports associated with the user 102. For example, the laboratory test reports associated with the user 102 may include, but are not limited to, Biometric/Biomarker's test reports (e.g., blood, urine, skin, saliva, etc.), Microbiome test reports (e.g., gut, virginal, oral microbiome, etc.), Genetic test reports (e.g., DNA and gene expression, etc.), and the like. The biometric test reports, and the microbiome and genetic test reports specify micronutrients and their dosages associated with the user 102. In one example, the external databases 118 may be databases of various external applications (not shown in FIGS.) that are capable of managing laboratory tests and storing the laboratory test reports of the user 102. In this case, the server system 116 retrieves the laboratory test reports associated with the user 102 through an application programming interface (API) rendered by corresponding external applications managing the external databases 118. In another example, the health application 124 may render options for allowing the user 102 to upload the laboratory test reports in the health application 124. The data extracted from the plurality of sources, the user survey response, and the laboratory test reports constitute the user information of the user 102.

At step 210, the server system 116 determines one or more micronutrients of the supplement based at least on the user information and the artificial intelligence (AI) model. In particular, the AI model associated with the server system 116 determines all the micronutrients present and recommended in the user profile and identifies any overlapping micronutrient dosage recommended from the laboratory test reports. Thereafter, the AI model determines appropriate micronutrients (i.e. the one or more micronutrients) to be incorporated in the supplement requested by the user 102 in current manufacturing. In some embodiment, the AI model associated with the server system 116 determines the micronutrients of the supplement to be manufactured for the user 102 based at least on one or more conditions. The conditions include the date of the laboratory test reports (for example, most recent lab test reports are considered as a high priority), Industry Upper Limits (UL) of each micronutrient, and recommended daily intake (RDI) of each micronutrient.

At step 212, the server system 116 determines one or more parameters associated with each micronutrient of the one or more micronutrients of the supplement. More specifically, the server system 116 determines one or more parameters associated with each micronutrient of the one or more micronutrients based at least on the user information and the AI model. The parameters associated with each micronutrient determined by the AI model may include, but are not limited to, an optimum dosage value, an optimal serving form, and an optimal serving frequency of the supplement. For example, the user 102 may request a supplement ‘ABC’. In this example scenario, the AI model associated with the server system 116 may determine the micronutrient to be incorporated in the supplement ‘ABC’ to be L-Theanine at an optimum dosage value of 77.38 milligrams. The AI model provides recommendations related to the optimum serving form (e.g., powdered for some micronutrients & softgel for other micronutrients) based on actual micronutrient raw properties, micronutrient source, maintaining bioavailability & optimal absorption by the human body. The AI model recommends the optimal serving frequency (e.g., 1 softgel in the morning with no food, 2 capsules around noon with food, 1 tablet before bed, etc.) of the supplement based on the user information as explained above.

At step 214, the server system 116 determines a time schedule for manufacturing and delivering the supplement to the user 102. More specifically, the server system 116 monitors the digital inventory of the user 102 to determine at least actual data pertaining to the micronutrient intake of the user 102, a quantity (or remaining servings) of the supplement. The server system 116 computes the time schedule to manufacture and ship (or deliver) the supplement requested by the user 102, based on determining the quantity of the supplement. In an embodiment, the user 102 is notified of the time schedule and/or expected time of arrival (ETA) in the health application 124 upon placing the request to manufacture the supplement through the health application 124.

At step 216, the server system 116 transmits supplement data and the time schedule to a manufacturing application 126 available on the user device 114 of the supplement manufacturer 112. The supplement data includes information related to the micronutrients of the supplement and the parameters associated with each micronutrient. Before transmitting the supplement data, the server system 116 identifies the supplement manufacturer 112 who can manufacture the supplement requested by the user 102 based on the recommendations provided by the AI model as explained above. For description purposes, only one supplement manufacturer is depicted in FIG. 1 who is associated with manufacturing the supplement for the user 102, and therefore it should not be taken to limit the scope of the present disclosure. Additionally, the server system 116 is configured to determine/forecast the amounts of raw materials to be sourced to the factory (i.e. the supplement manufacturer 112) ahead of time based on actual/anticipated users' consumptions in order to meet manufacturing needs all the time.

At step 218, the supplement manufacturer 112 manufactures the supplement according to the recommendations of the AI model. At step 220, the supplement is shipped to the user 102 in a timely manner (i.e. just-in-time manufacturing and delivering the supplement). In other words, the supplement is shipped to the user 102 as determined in the time schedule (or before the expiry of the ETA) to prevent gaps in the consumption habits of the user 102. It is to be understood that the server system 116 transmits user communication details to the manufacturing application 126, thereby enabling the supplement manufacturer 112 to ship the supplement to the user 102. It is evident that the server system 116 allows optimal manufacturing of the supplement unique to each user (i.e. the user 102) based at least on the user information. As such, the process of optimal manufacturing of the supplement unique to each user conforms to a dynamic manufacturing process.

In an embodiment, the user 102 may scan a unique code (not shown in FIGS.) of the supplement upon delivery of the supplement for determining the authenticity of the supplement. More specifically, the user 102 scans the unique code of the supplement using the user device 104 and transmits the unique code to the server system 116 through the health application 124 for determining the authenticity of the supplement. The server system 116, by accessing a manufacturer supply chain network, performs authentication of the supplement i.e. if the supplement is manufactured and delivered from reliable/trusted sources, the ingredients and certifications are legit and the like. Upon determining the authenticity, the user 102 may consume the supplement in order to accomplish the nutrition goal determined for the user 102 in the health application 124.

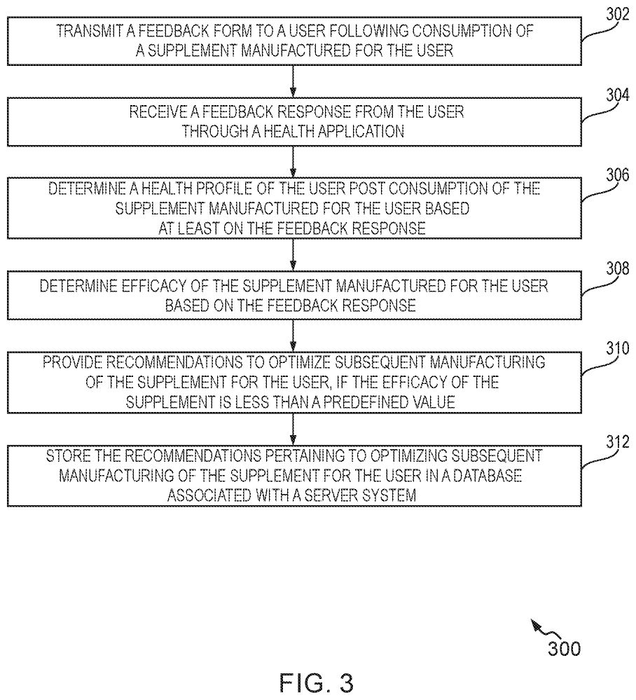

FIG. 3 represents a process flow 300 for optimizing subsequent manufacturing of the supplement for the user 102 via a feedback loop facilitated through the health application 124, in accordance with an embodiment of the present disclosure. The sequence of operations of the process flow 300 may not be necessarily executed in the same order as they are presented. Further, one or more operations may be grouped and performed in the form of a single step, or one operation may have several sub-steps that may be performed in parallel or a sequential manner.

At 302, the server system 116 transmits a feedback form to the user 102 following the consumption of the supplement manufactured for the user 102. As explained above, the server system 116 monitors the digital inventory of the user 102 to determine the intake of the supplement manufactured for the user 102. Upon determining the intake of the supplement, the server system 116 transmits the feedback form to the user device 104. The feedback form transmitted to the user 102 may include various data fields for receiving inputs related to the health profile of the user 102 following the consumption of the supplement which will be explained further in detail.

At 304, the server system 116 receives a feedback response from the user 102 through the health application 124. The feedback form including the user inputs in various data fields corresponds to the feedback response. The feedback response may include health and wellbeing information of the user 102, the laboratory test reports of the user 102, and data from the nutrient tracker 108 and the wearable device 106 post consumption of the supplement. It is to be noted that feedback response including the health and wellbeing information, the laboratory test reports, and the data from the nutrient tracker 108 and the wearable device 106 acts as a clinical trial for the supplement.

At 306, the server system 116 determines the health profile of the user 102 post consumption of the supplement manufactured for the user 102 based at least on the feedback response. More specifically, the server system 116 utilizes the laboratory test reports, and the data from the nutrient tracker 108 and the wearable device 106 to determine the health profile of the user 102 post consumption of the supplement by the user 102. In other words, server system 116 determines at least the actual micronutrients availability levels in the user's body, the micronutrients dosages/forms consumed on the day of the laboratory tests, the efficiency of the serving, and the dosages/forms of micronutrients included in achieving the nutrition goal based at least on the feedback response.

At 308, the server system 116 determines the efficacy of the supplement manufactured for the user 102 based at least on the feedback response.

At 310, the server system 116 provides recommendations to optimize subsequent manufacturing of the supplement for the user 102, if the efficacy of the supplement is less than a predefined value. In particular, the server system 116 determines whether to update/improve a certain recommendation/manufacturing of the supplement for the user 102 at future instances based, at least in part, on the feedback response and the AI model. The recommendations for optimizing subsequent manufacturing of the supplement include information related to optimum dosage values and serving form to be selected for each micronutrient of the supplement requested by the user 102 in the future and/or subsequent manufacturing process. Thus, optimizing future recommendations and/or subsequent manufacturing of the supplement based at least on the feedback loop (i.e. the feedback response) corresponds to the smart formulation. In other words, the server system 116 is able to smart formulate the supplements in specific nutrients dosages in a specific serving form in specific per serving-nutrients combo to the user's unique body based at least on the feedback response.

At 312, the server system 116 stores the recommendations pertaining to optimizing subsequent manufacturing of the supplement for the user 102 in a database associated with the server system 116.

Further, the smart formulation technique employed by the server system 116 to optimize subsequent manufacturing of the supplement for the user 102 is considered to be unbiased. In particular, the server system 116 is configured to track the intake of random supplements and determine their impact on the user 102 from the laboratory test reports, and the data from the nutrient tracker 108, and the wearable device 106. The AI model associated with the server system 116 utilizes the feedback response following the consumption of the supplement and the impact of the random supplements on the user's body for optimizing subsequent manufacturing of the supplement for the user 102.

In an embodiment, the server system 116 may be configured to establish generic and unestablished daily values in percentage (% DVs) of micronutrients for a certain user profile. Currently, very few of the thousands of micronutrients have established daily values (DVs) listed on the nutrition labels and are still based on a generic 2,000 calories diet. Further, the established DVs associated with the micronutrients are not updated as not all of the micronutrients are widely consumed. The server system 116 is configured to identify such micronutrients by utilizing the laboratory reports for determining the % DVs of micronutrients.

FIG. 4 illustrates an example representation of a user interface (UI) 400, depicting a request to manufacture the supplement initiated by the user 102 to the server system 116 through the health application 124, in accordance with an embodiment of the present disclosure. The user 102 may be prompted with the UI 400 for transmitting the request to manufacture the supplement, upon providing inputs in the health application 124.

The UI 400 is depicted to include a title 402 associated with the text ‘SUPPLEMENT INFORMATION’ and a supplement information section 404. The supplement information section 404 is depicted to include details such as but not limited to, a supplement name 404a, serving form 404b, serving amounts 404c, and supplement functionality information 404d. The supplement name 404a, the serving form 404b, the serving amounts 404c and the supplement functionality information 404d are exemplarily depicted to be ‘Vitamin B complex’, ‘powder’, ‘2 scoops’ and ‘Immune support & antioxidant’, respectively. Further, the UI 400 is depicted to include a data field 406 for receiving input related to a quantity of the supplement (exemplarily depicted to be ‘1 no.’). The UI 400 is further depicted to include a nutrients section 408. The nutrients section 408 is depicted to include micronutrients (exemplarily depicted to be ‘riboflavin’, ‘niacin’, and ‘pantothenic acid’) of the supplement.

Further, the UI 400 is depicted to include a data field 410. The user 102 may provide user preferences related to the supplement 404a such as, but not limited to, serving form, flavoring agents preferred by the user 102, functionality, and the like. For example, the UI 400 is depicted to include the serving form as ‘powder’ for the supplement 404a. In one instance, the user (such as the user 102) may be an athlete who would like to consume the supplement 404a in other formats (or serving form) such as ready-to-drink form or quick-to-consume like gummies. Further the user 102 would like a certain flavor say blueberries and the functionality to be sustained energy without added caffeine, and the like. The user 102 may describe and/or provide inputs in the data field 410 related to the serving format, functionality, flavoring agents, etc. Thereafter, the user 102 transmits the request to the server system 116 by providing an input (e.g., click or tap) on a button 412 rendered in the UI 400, upon entering the details pertaining to the supplement, the quantity of the supplement, etc. The server system 116 determines the micronutrients and their appropriate dosages based at least on the inputs provided in the request as explained with reference to FIG. 2.

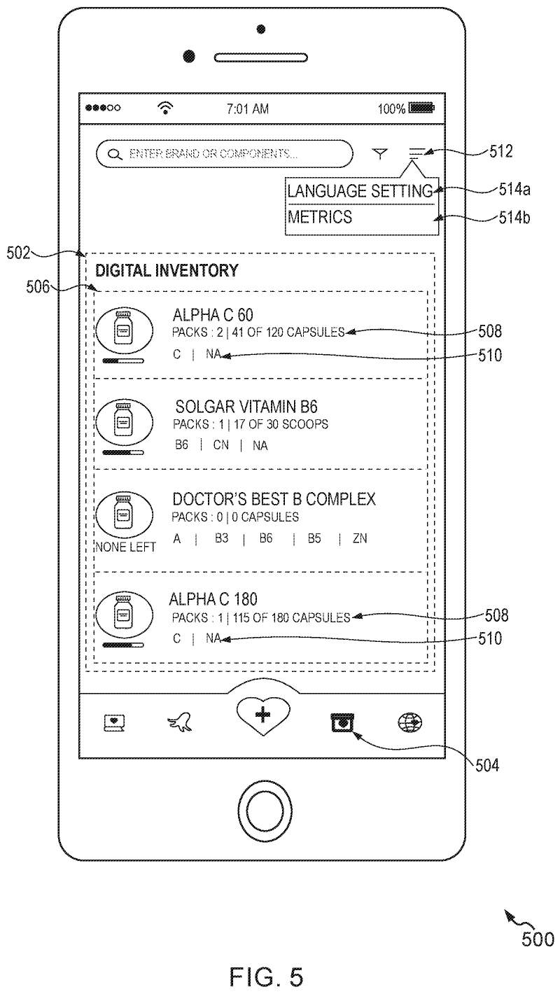

FIG. 5 illustrates an example representation of a user interface (UI) 500, depicting a digital inventory associated with the user 102 in the health application 124, in accordance with an embodiment of the present disclosure. The UI 500 depicts a digital inventory 502 based on a user input on an actionable icon 504.

The digital inventory 502 of the UI 500 is depicted to include a plurality of supplements 506 owned by the user 102. The UI 500 further depicts a quantity 508 and micronutrients 510 associated with each supplement. As explained above, the health application 124 renders the list of customizable settings to the user 102, for allowing the user 102 to customize and/or personalize the digital inventory 502 as per user preference. More specifically, the user 102 is rendered with a pop-up menu including a list of customizable settings upon user selection of a button 512. The list of customizable settings includes a customizable setting 514a and a customizable setting 514b. The customizable settings 514a and 514b are associated with the text ‘LANGUAGE SETTING’, and ‘METRICS’, respectively. The user 102 may translate the language of choice (e.g., English to German) by providing input on the customizable setting 514a. Upon providing the input on the customizable setting 514a, the user 102 may be rendered with a UI (not shown in FIGS.) depicting a list of options for user selection of the language. Further, the user 102 may convert all metrics of dosage values associated with each micronutrient 510 of the supplements 506 (e.g., converting all metrics to a unified unit ‘milligram’ (mg) across all micronutrients). Upon providing the input on the customizable setting 514b, the user 102 may be rendered with a UI (not shown in FIGS.) depicting a list of options for user selection of the metrics.

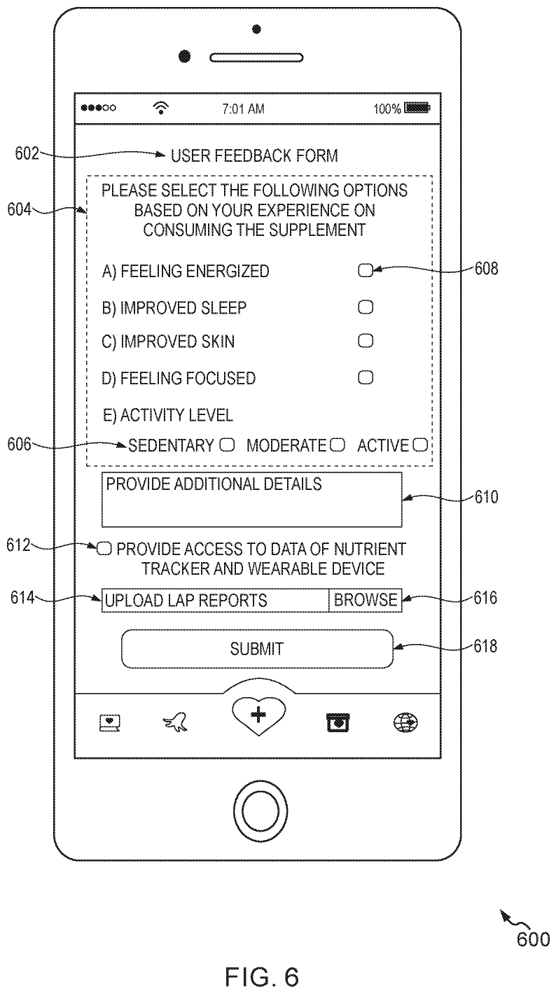

FIG. 6 illustrates an example representation of a user interface (UI) 600, depicting a feedback form, in accordance with an embodiment of the present disclosure. The UI 600 is depicted to include a feedback form 602 rendered in the health application 124. As explained above, the feedback form 602 is transmitted to the user device 104 of the user 102 following consumption of the supplement (i.e. the supplement 404a) manufactured for the user 102.

The UI 600 is depicted to include a list of options 604 for receiving inputs related to user experience (or health and wellbeing information) upon consumption of the supplement (i.e. the supplement 404a) manufactured for the user 102. The list of options 604 is exemplarily depicted to be ‘feeling energized’, ‘improved sleep’, ‘improved skin’, ‘feeling focused’, and ‘activity level’. As shown in FIG. 6, the option ‘activity level’ includes one or more sub-options 606 (exemplarily depicted to be ‘sedentary’, ‘moderate’, and ‘active’). Further, each option and the sub-option listed in the UI 600 is associated with a radio button 608. The user 102 may provide inputs on the corresponding radio button 608 for selecting the user experience upon consumption of the supplement (i.e. the supplement 404a) by the user 102. Further, the UI 600 is depicted to include a data field 610. The user 102 can describe the user experience by providing additional information following the consumption of the supplement (i.e. the supplement 404a) in the data field 610. The UI 600 is further depicted to include a dialog box 612 for receiving approval from the user 102 to access and/or utilize the data from the nutrient tracker 108 and the wearable device 106 as explained with reference to FIG. 3. Further, the inputs in the data field 610 and the dialog box 612 may not be mandatory and/or are optional. In one scenario, a user and/or a consumer, such as the user 102 may not own the gadgets (i.e. the nutrient tracker 108 and the wearable device 106). In this scenario, the user 102 may skip providing the inputs in the dialog box 612. Further, the server system 116 determines the information that is detected by the nutrient tracker 108 and the wearable device 106 for the user 102 based on the user profiles of the other users available in the database associated with the server system 116 which is explained with reference to FIG. 2.

The UI 600 depicts an input field 614 for providing the laboratory test reports associated with the user 102. The laboratory test reports correspond to the test reports of the lab tests conducted after consumption of the supplement (i.e. the supplement 404a). The user 102 provides input on a button 616 for manually uploading the laboratory test reports in the health application 124. Upon providing the input on the button 616, the user 102 may be directed to a UI (not shown in FIGS.) for selecting the laboratory test reports from a database (not shown in FIGS.) associated with the user device 104, or for selecting from external databases (i.e. the external databases 118). In an embodiment, the server system 116 is configured to automatically extract the laboratory test reports from the external databases 118 and data from the nutrient tracker 108 and the wearable device 106 as explained above. Thereafter, the feedback response (i.e. the feedback form 602 including user inputs) is transmitted to the server system 116 based on providing input on a button 618. The options listed in the list of options 604 are selected for description purposes, and therefore they should not be considered for limiting the scope of the present disclosure. The server system 116 determines the efficacy of the supplements manufactured for the user 102 based at least on the user inputs provided in the feedback form (i.e. response).

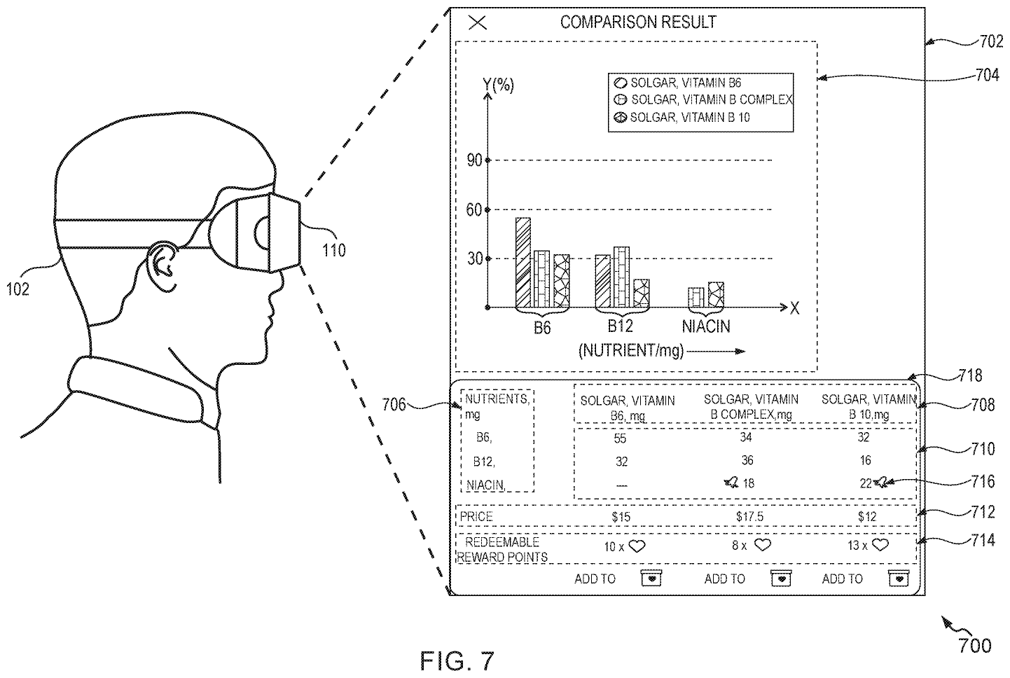

FIG. 7 represents an example scenario 700 depicting a comparison of supplements rendered on the XR device 110 associated with the user 102, in accordance with an embodiment of the present disclosure. As explained above, the XR device 110 is communicably coupled to the user device 104. The health application 124 renders a set of features (and/or XR settings) for establishing a connection with the XR device 110 of the user 102.

As shown in FIG. 7, a comparison result 702 is rendered on the XR device 110 of the user 102. The comparison result 702 includes information related to the comparison of two or more supplements (see, 708). For example, a user/consumer (i.e. the user 102) is shopping in the health application 124 and selects the two or more supplements 708 (exemplarily depicted to be ‘Solgar, Vitamin B6’, ‘Solgar, Vitamin B complex’ and ‘Solgar, Vitamin B10’) from the supplements offered in the health application 124 and runs a comparison on the health application 124 in order to purchase a suitable supplement. The health application 124 provides the comparison result 702 by comparing the two or more supplements 708 on an ingredient level (or micronutrient level).

In particular, the comparison result 702 of the two or more supplements (see, 708) are depicted on a comparison graph 704 (e.g., a bar graph) and a comparison table 718. The comparison table 718 is depicted to include a list of micronutrients 706 associated with each supplement of the two or more supplements 708 selected by the user 102. Further, the UI 700 is depicted to include dosage values 710 for each micronutrient of the list of micronutrient 706. Thus, it is evident that the comparison table 718 provides a comparison of each of the supplement 708 on a micronutrient level or ingredient level. For example, the dosage values of the nutrient 136′ are depicted to be ‘55 milligram (mg)’, ‘34 mg’ and ‘32 mg’. Further, the comparison table 718 is depicted to include a price 712 and reward points 714 for each of the two or more supplements 708 depicted in the comparison table 718. The price 712 is the gross price of each of the two or more supplements 708. Further, the reward points 714 may be the reward points in form of digital currency (i.e. crypto-collectibles) provided to the user 102 as explained above. As such, the health application 124 determines the reward points 714 in form of digital currency that can be redeemed on each supplement of the two or more supplements 708.

Additionally, the comparison table 718 is depicted to include an indicator 716 in the form of an alert message. More specifically, the server system 116 is configured to determine if at least one micronutrient from the list of micronutrients 706 is incompatible for the user 102 based, at least in part, on the user information extracted from the plurality of sources and the external databases 118 periodically. Thereafter, the server system 116 is configured to display the alert message in form of the indicator (see, 716) corresponding to the at least one micronutrient in the health application 124. The indicator 716 may be an indication that the at least one micronutrient may be synthetic, non-biological, or allergic ingredients.

The comparison graph 704 includes each micronutrient from the list of micronutrients 706 associated with each of the two or more supplements 708 represented on the X-axis and the dosage values 710 of each micronutrient represented on the Y-axis (e.g., as shown in FIG. 7). As such, the comparison graph 704 is plotted by aligning the same micronutrients of the supplements 708 on the same line with each bar representing the micronutrient associated with each supplement of the two or more supplements 708. For example, the dosage values of a micronutrient (e.g., B6), offered by each supplement of the two or more supplements 708 are plotted using different bars in the comparison graph 704. Similarly, the dosage values of other micronutrients (e.g., B12 and Niacin) are plotted in the comparison graph 704. In one embodiment, each bar in the comparison graph 704 may be uniquely colored (not shown in figures). As such, this form of representation of the micronutrients 706 provides easy comprehension to the user 102 to compare. Thus, the comparison result 702 including the comparison table 718 and the comparison graph 704 rendered on the XR device 110 allows the user 102 to compare and decide the best suitable product for purchase.

In an embodiment, the health application 124 managed by the server system 116 is configured to extract information of a supplement (i.e. the supplement 404a) by scanning the nutrition label of the supplement using the user device 104 or the XR device 110. In another embodiment, the health application 124 may retrieve information about the supplement by scanning a quick response (QR) code associated with the supplement.

In one embodiment, the supplement may be integrated with near-field communication (NFC) technology. As such, the user 102 can import information of the supplement to the health application 124 based at least on NFC associated with the user device 104. In other words, the user 102 can tap the user device 104 on the supplement to import the information of the supplement to the health application 124.

In an embodiment, scanning through the health application 124 can retrieve supplement information, authenticate supplement, and the micronutrients as well as sources by being connected to a supply blockchain network. Thus, allowing the user 102 to authenticate the supplement purchased and/or about to be purchased from the two or more supplements 708 in the health application 124.

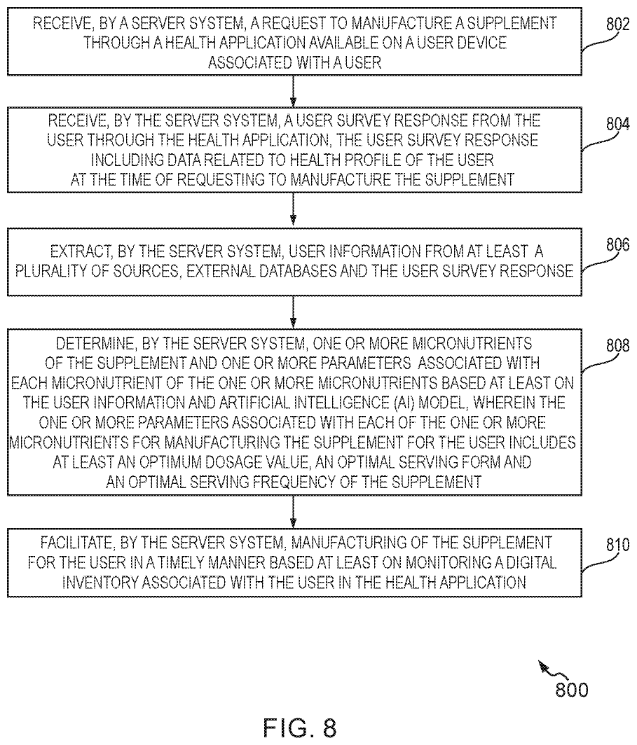

FIG. 8 illustrates a flow diagram of a computer-implemented method 800 for facilitating dynamic manufacturing of the supplement for the user 102, in accordance with an embodiment of the present disclosure. The method 800 depicted in the flow diagram may be executed by, for example, the server system 116. Operations of the flow diagram of the method 800, and combinations of operation in the flow diagram of the method 800, may be implemented by, for example, hardware, firmware, a processor, circuitry, and/or a different device associated with the execution of software that includes one or more computer program instructions. It is noted that the operations of the method 800 can be described and/or practiced by using a system other than these server systems. The method 800 starts at operation 802.

At operation 802, the method 800 includes receiving, by the server system 116, a request to manufacture a supplement through a health application 124 available on a user device 104 associated with the user 102.

At operation 804, the method 800 includes receiving, by the server system 116, a user survey response through the health application 124. The user survey response includes data related to the health profile of the user 102 at the time of requesting to manufacture the supplement. The user survey is transmitted to the user device 104 upon receipt of the request from the user 102 for manufacturing the supplement as explained with reference to FIG. 2. The user 102 provides inputs in the user survey which determines the health profile of the user 102 at the time of requesting to manufacture the supplement. The user survey including the inputs corresponds to the user survey response.

At operation 806, the method 800 includes extracting, by the server system 116, user information from at least a plurality of sources, external databases 118, and user survey responses through the health application 124.

At operation 808, the method 800 includes determining, by the server system 116, one or more micronutrients of the supplement and one or more parameters associated with each micronutrient of the one or more micronutrient based at least on the user information and artificial intelligence (AI) model. The one or more parameters associated with each of the one or more micronutrients for manufacturing the supplement for the user 102 includes at least an optimum dosage value, an optimal serving form, and an optimal serving frequency of the supplement.

At operation 810, the method 800 includes facilitating, by the server system 116, manufacturing of the supplement for the user 102 in a timely manner based at least on monitoring a digital inventory (see, 502) associated with the user 102 in the health application 124.

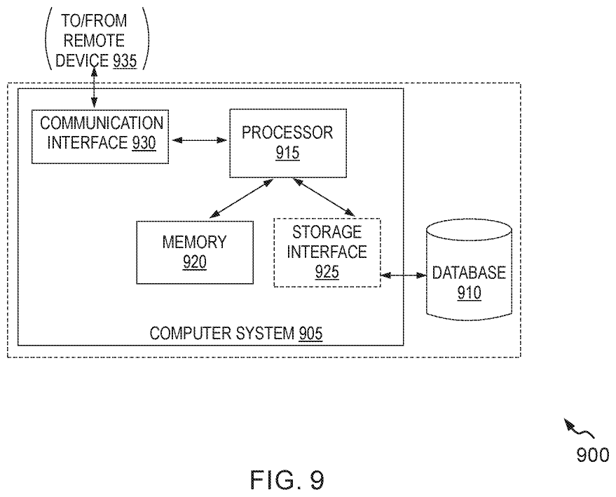

FIG. 9 is a simplified block diagram of a server system 900 for facilitating dynamic manufacturing of the supplement (e.g., the supplement 404a) to the user 102, in accordance with one embodiment of the present disclosure. The server system 900 is an example of the server system 116 of FIG. 1. In one example embodiment, the server system 900 may be a separate part and may operate apart via the network 122. In some embodiments, the server system 900 is embodied as a cloud-based and/or SaaS-based (software as a service) architecture. Further, the server system 900 includes a computer system 905 and a database 910.

The computer system 905 includes at least one processor 915 for executing instructions. Instructions may be stored in, for example, but not limited to, a memory 920. The processor 915 may include one or more processing units (e.g., in a multi-core configuration). Examples of the processor 915 include, but are not limited to, an application-specific integrated circuit (ASIC) processor, a reduced instruction set computing (RISC) processor, a complex instruction set computing (CISC) processor, a field-programmable gate array (FPGA), and the like. The memory 920 includes suitable logic, circuitry, and/or interfaces to store a set of computer-readable instructions for performing operations. Examples of the memory 920 include a random-access memory (RAM), a read-only memory (ROM), a removable storage drive, a hard disk drive (HDD), and the like. It will be apparent to a person skilled in the art that the scope of the disclosure is not limited to realizing the memory 920 in the server system 900, as described herein. In another embodiment, the memory 920 may be realized in the form of a database server or cloud storage working in conjunction with the server system 900, without departing from the scope of the present disclosure.

The processor 915 is operatively coupled to a communication interface 930 such that computer system 905 is capable of communicating with a remote device 935 such as the user devices 104 and 114, the nutrient tracker 108, the wearable device 106, the XR device 110, the blockchain network 120, external databases 118, and the like. For example, the communication interface 930 may receive the set of data inputs, feedback responses, user surveys, authentication requests, and the like.

The processor 915 may also be operatively coupled to the database 910. The database 910 is any computer-operated hardware suitable for storing and/or retrieving data, such as, but not limited to, the digital inventory, supplements information, user profiles, and the like. In some embodiment, the database 910 is integrated within the computer system 905. For example, the database 910 may include multiple storage units such as hard disks and/or solid-state disks in a redundant array of inexpensive disks (RAID) configuration. The database 910 may include a storage area network (SAN) and/or a network-attached storage (NAS) system. In other embodiments, the database 910 is external to the computer system 905 and may be accessed by the computer system 905 using a storage interface 925. The storage interface 925 is any component capable of providing the processor 915 with access to the database 910. The storage interface 925 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 915 with access to the database 910.

In an embodiment, the processor 915 is configured to receive a request to manufacture a supplement for the user 102. The processor 915 extracts user information related to the user 102 from various data sources. The user information extracted from various data sources corresponds to input data. The input data is utilized by the (AI) model associated with the processor 915 to determine the nutrients and their optimum dosage values, serving form, and supplement frequency suitable for the user 102. The processor 915 is configured to utilize the latest available information as the input data for performing the aforementioned function. Thereafter, the processor 915 determines the time schedule for manufacturing and shipping the supplement to the user 102. Further, the processor 915 is configured to authenticate the supplement for the user 102 based at least on accessing the manufacturing supply chain network. The one or more operations performed by the server system 900 are already explained with references to FIGS. 1-8, and they are not reiterated herein for the sake of brevity.

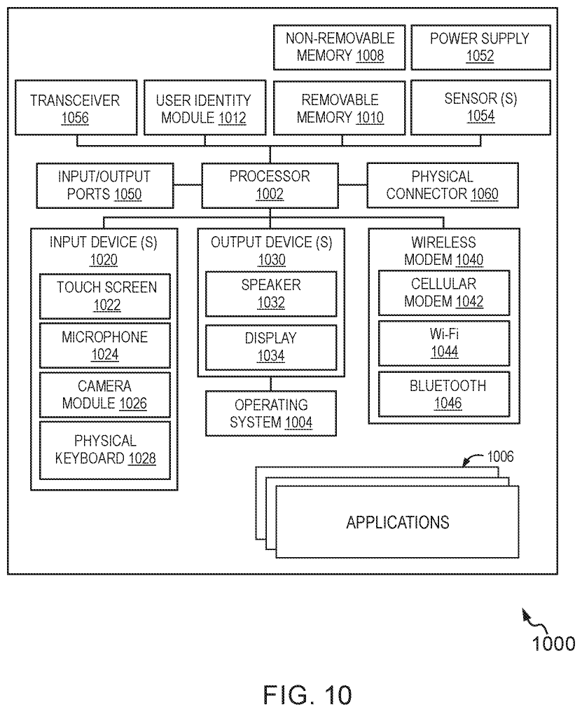

FIG. 10 is a simplified block diagram of an electronic device 1000 capable of implementing various embodiments of the present disclosure. For example, the electronic device 1000 may correspond to the user device 104 or the user device 114 of FIG. 1. The electronic device 1000 is depicted to include one or more applications 1006. For example, the one or more applications 1006 may include the health application 124 or the manufacturing application 126 of FIG. 1. The health application 124 and the manufacturing application 126 can be an instance of an application downloaded from the server system 900 or the server system 116. One of the applications 1006 installed on the electronic device 1000 is capable of communicating with a server system for managing the nutrient intake of the user 102.

It should be understood that the electronic device 1000 as illustrated and hereinafter described is merely illustrative of one type of device and should not be taken to limit the scope of the embodiments. As such, it should be appreciated that at least some of the components described below in connection with the electronic device 1000 may be optional and thus in an embodiment may include more, less, or different components than those described in connection with the embodiment of FIG. 10. As such, among other examples, the electronic device 1000 could be any of a mobile electronic device, for example, cellular phones, tablet computers, laptops, mobile computers, personal digital assistants (PDAs), mobile televisions, mobile digital assistants, or any combination of the aforementioned, and other types of communication or multimedia devices.

The illustrated electronic device 1000 includes a controller or a processor 1002 (e.g., a signal processor, microprocessor, ASIC, or other control and processing logic circuitry) for performing such tasks as signal coding, data processing, image processing, input/output processing, power control, and/or other functions. An operating system 1004 controls the allocation and usage of the components of the electronic device 1000 and supports for one or more operations of the application (see, the applications 1006), such as the application 124 and the application 126 that implements one or more of the innovative features described herein. In addition, the applications 1006 may include common mobile computing applications (e.g., telephony applications, email applications, calendars, contact managers, web browsers, messaging applications) or any other computing application.

The illustrated electronic device 1000 includes one or more memory components, for example, a non-removable memory 1008 and/or removable memory 1010. The non-removable memory 1008 and/or the removable memory 1010 may be collectively known as a database in an embodiment. The non-removable memory 1008 can include RAM, ROM, flash memory, a hard disk, or other well-known memory storage technologies. The removable memory 1010 can include flash memory, smart cards, or a Subscriber Identity Module (SIM). The one or more memory components can be used for storing data and/or code for running the operating system 1004 and the applications 1006. The electronic device 1000 may further include a user identity module (UIM) 1012. The UIM 1012 may be a memory device having a processor built in. The UIM 1012 may include, for example, a subscriber identity module (SIM), a universal integrated circuit card (UICC), a universal subscriber identity module (USIM), a removable user identity module (R-UIM), or any other smart card. The UIM 1012 typically stores information elements related to a mobile subscriber. The UIM 1012 in form of the SIM card is well known in Global System for Mobile (GSM) communication systems, Code Division Multiple Access (CDMA) systems, or with third-generation (3G) wireless communication protocols such as Universal Mobile Telecommunications System (UMTS), CDMA9000, wideband CDMA (WCDMA) and time division-synchronous CDMA (TD-SCDMA), or with fourth-generation (4G) wireless communication protocols such as LTE (Long-Term Evolution).

The electronic device 1000 can support one or more input devices 1020 and one or more output devices 1030. Examples of the input devices 1020 may include, but are not limited to, a touch screen/a display screen 1022 (e.g., capable of capturing finger tap inputs, finger gesture inputs, multi-finger tap inputs, multi-finger gesture inputs, or keystroke inputs from a virtual keyboard or keypad), a microphone 1024 (e.g., capable of capturing voice input), a camera module 1026 (e.g., capable of capturing still picture images and/or video images) and a physical keyboard 1028. Examples of the output devices 1030 may include, but are not limited to, a speaker 1032 and a display 1034. Other possible output devices can include piezoelectric or other haptic output devices. Some devices can serve more than one input/output function. For example, the touch screen 1022 and the display 1034 can be combined into a single input/output device.

A wireless modem 1040 can be coupled to one or more antennas (not shown in FIG. 10) and can support two-way communications between the processor 1002 and external devices, as is well understood in the art. The wireless modem 1040 is shown generically and can include, for example, a cellular modem 1042 for communicating at long range with the mobile communication network, a Wi-Fi compatible modem 1044 for communicating at short range with an external Bluetooth-equipped device, or a local wireless data network or router, and/or a Bluetooth-compatible modem 1046. The wireless modem 1040 is typically configured for communication with one or more cellular networks, such as a GSM network for data and voice communications within a single cellular network, between cellular networks, or between the electronic device 1000 and a public switched telephone network (PSTN).

The electronic device 1000 can further include one or more input/output ports 1050, a power supply 1052, one or more sensors 1054 for example, an accelerometer, a gyroscope, a compass, or an infrared proximity sensor for detecting the orientation or motion of the electronic device 1000 and biometric sensors for scanning biometric identity of an authorized user, a transceiver 1056 (for wirelessly transmitting analog or digital signals) and/or a physical connector 1000, which can be a USB port, IEEE 1294 (FireWire) port, and/or RS-232 port. The illustrated components are not required or all-inclusive, as any of the components shown can be deleted and other components can be added.

The disclosed method with reference to FIG. 8, or one or more operations of the server system 116 may be implemented using software including computer-executable instructions stored on one or more computer-readable media (e.g., non-transitory computer-readable media, such as one or more optical media discs, volatile memory components (e.g., DRAM or SRAM), or non-volatile memory or storage components (e.g., hard drives or solid-state non-volatile memory components, such as Flash memory components)) and executed on a computer (e.g., any suitable computer, such as a laptop computer, net book, Web book, tablet computing device, smart phone, or other mobile computing device). Such software may be executed, for example, on a single local computer or in a network environment (e.g., via the Internet, a wide-area network, a local-area network, a remote web-based server, a client-server network (such as a cloud computing network), or other such network) using one or more network computers. Additionally, any of the intermediate or final data created and used during implementation of the disclosed methods or systems may also be stored on one or more computer-readable media (e.g., non-transitory computer-readable media) and are considered to be within the scope of the disclosed technology. Furthermore, any of the software-based embodiments may be uploaded, downloaded, or remotely accessed through a suitable communication means. Such a suitable communication means includes, for example, the Internet, the World Wide Web, an intranet, software applications, cable (including fiber optic cable), magnetic communications, electromagnetic communications (including RF, microwave, and infrared communications), electronic communications, or other such communication means.

Although the invention has been described with reference to specific exemplary embodiments, it is noted that various modifications and changes may be made to these embodiments without departing from the broad spirit and scope of the invention. For example, the various operations, blocks, etc., described herein may be enabled and operated using hardware circuitry (for example, complementary metal oxide semiconductor (CMOS) based logic circuitry), firmware, software and/or any combination of hardware, firmware, and/or software (for example, embodied in a machine-readable medium). For example, the apparatuses and methods may be embodied using transistors, logic gates, and electrical circuits (for example, application specific integrated circuit (ASIC) circuitry and/or in Digital Signal Processor (DSP) circuitry).

Particularly, the server system 116 and its various components may be enabled using software and/or using transistors, logic gates, and electrical circuits (for example, integrated circuit circuitry such as ASIC circuitry). Various embodiments of the invention may include one or more computer programs stored or otherwise embodied on a computer-readable medium, wherein the computer programs are configured to cause a processor of the computer to perform one or more operations. A computer-readable medium storing, embodying, or encoded with a computer program, or similar language may be embodied as a tangible data storage device storing one or more software programs that are configured to cause a processor or computer to perform one or more operations. Such operations may be, for example, any of the steps or operations described herein. In some embodiments, the computer programs may be stored and provided to a computer using any type of non-transitory computer-readable media. Non-transitory computer-readable media include any type of tangible storage media. Examples of non-transitory computer-readable media include magnetic storage media (such as floppy disks, magnetic tapes, hard disk drives, etc.), optical magnetic storage media (e.g., magneto-optical disks), CD-ROM (compact disc read-only memory), CD-R (compact disc recordable), CD-R/W (compact disc rewritable), DVD (Digital Versatile Disc), BD (BLU-RAY® Disc), and semiconductor memories (such as mask ROM, PROM (programmable ROM), EPROM (erasable PROM), flash memory, RAM (random access memory), etc.). Additionally, a tangible data storage device may be embodied as one or more volatile memory devices, one or more non-volatile memory devices, and/or a combination of one or more volatile memory devices and non-volatile memory devices. In some embodiments, the computer programs may be provided to a computer using any type of transitory computer-readable media. Examples of transitory computer-readable media include electric signals, optical signals, and electromagnetic waves. Transitory computer-readable media can provide the program to a computer via a wired communication line (e.g., electric wires, and optical fibers) or a wireless communication line.

Various embodiments of the disclosure, as discussed above, may be practiced with steps and/or operations in a different order, and/or with hardware elements in configurations, which are different than those which are disclosed. Therefore, although the disclosure has been described based upon these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

1. A computer-implemented method, comprising:
receiving, by a server system, a request to manufacture a supplement through a health application available on a user device associated with a user;
upon receipt of the request, transmitting a user survey to the user device;
receiving, by the server system, a user survey response from the user through the health application, the user survey response comprising data related to a health profile of the user at the time of requesting to manufacture the supplement;
extracting, by the server system, user information from at least a plurality of sources, external databases, and the user survey response;
determining, by the server system, one or more micronutrients to be incorporated in the supplement and one or more parameters associated with each micronutrient of the one or more micronutrients based at least on the user information and artificial intelligence (AI) model,
wherein the one or more parameters associated with each of the one or more micronutrients for manufacturing the supplement for the user comprises at least an optimum dosage value, an optimal serving form, and an optimal serving frequency of the supplement;
computing, by the server system, the time schedule to manufacture and deliver the supplement requested by the user by monitoring the digital inventory of the user and the user is notified of the time schedule or expected time of arrival of the supplement in the health application upon receiving the request to manufacture the supplement through the health application from the user; and
facilitating, by the server system, manufacturing and delivery of the supplement_{—} with the one or more micronutrients according to the recommendations of the AI model for the user in a timely manner based at least on monitoring a digital inventory associated with the user in the health application;
determining, by the server system, the authenticity of the delivered supplement based on a unique code of the supplement, wherein the unique code of the supplement is scanned and transmitted using the user device to the server system through the health application.

2. The computer-implemented method as claimed in claim 1, wherein the user information is extracted on a periodic basis from the plurality of sources and the external databases for manufacturing the supplement, and wherein the user information comprises data related to a plurality of supplements comprised in the digital inventory, health metrics, dosage values, serving form, and quantity of each micronutrient available in the user's body.

3. The computer-implemented method as claimed in claim 1, wherein the plurality of sources comprises a user profile of the user in the health application, the user device, and one or more wearable devices and nutrient trackers associated with the user communicably coupled to the health application, and wherein the external databases comprise one or more laboratory test reports associated with the user.

4. The computer-implemented method as claimed in claim 1, wherein facilitating manufacturing of the supplement for the user further-comprises:
determining, by the server system, a time schedule to manufacture and deliver the supplement to the user based, at least in part, on the digital inventory associated with the user; and
transmitting, by the server system, supplement data comprising the one or more micronutrients of the supplement and the one or more parameters associated with each micronutrient, and the time schedule to a manufacturing application available on a user device associated with a supplement manufacturer.

5. The computer-implemented method as claimed in claim 1, further comprising:
performing, by the server system, authentication of the supplement comprising the one or more micronutrients that are manufactured for the user based, at least in part, on a manufacturing supply chain network.

6. The computer-implemented method as claimed in claim 1, further comprising:
receiving, by the server system, a feedback response from the user through the health application following consumption of the supplement manufactured for the user for achieving a nutrition goal determined for the user in the health application;
determining, by the server system, efficacy of the supplement manufactured for the user based, at least in part, on the feedback response; and
providing, by the server system, recommendations to optimize subsequent manufacturing of the supplement for the user, if the efficacy of the supplement on the user is less than a predefined value, wherein the recommendations for optimizing subsequent manufacturing of the supplement comprises information related to optimum dosage values and serving form to be selected for each micronutrient of the supplement.

7. The computer-implemented method as claimed in claim 1, further comprising:
providing, by the server system, reward points in form of digital currency to the user based, at least in part, on consumption habits of the user, purchasing supplements in the health application and user activities in the health application, wherein the reward points in the form of digital currency provided to the user are crypto collectibles.

8. The computer-implemented method as claimed in claim 1, further comprising:
rendering, by the server system, a set of features in the health application for establishing a connection between an extended reality (XR) device associated with the user and the health application available on the user device; and
facilitating, by the server system, display of a comparison result of two or more supplements on the XR device of the user, wherein the comparison result provides a comparison of the two or more supplements on an ingredient level comprising a list of micronutrients associated with each of the two or more supplements, thereby allowing the user to purchase a suitable supplement from the two or more supplements available in the health application.

9. The computer-implemented method as claimed in claim 8, further comprising:
determining, by the server system, at least one micronutrient from the list of micronutrients is incompatible for the user based, at least in part, on the user information extracted from the plurality of sources and the external databases on a periodic basis; and
upon determining the at least one micronutrient is incompatible for the user, facilitating, by the server system, display of an alert message in form of an indicator corresponding to the at least one micronutrient in the health application.

10. The computer-implemented method as claimed in claim 1, further comprising:
rendering, by the server system, a list of customizable settings in the health application for allowing the user to customize the digital inventory of the user on an ingredient level in the health application.

11. A server system, comprising:
a communication interface;
a memory comprising executable instructions; and
a processor communicably coupled to the communication interface and the memory, the processor configured to cause the server system to perform, at least in part, to:
receive a request to manufacture a supplement through a health application available on a user device associated with a user,
upon receipt of the request, transmit a user survey to the user device
receive a user survey response from the user through the health application, the user survey response comprising data related to a health profile of the user at the time of requesting to manufacture the supplement,
extract user information from at least a plurality of sources, external databases and the user survey response,
determine one or more micronutrients to be incorporated in the supplement and one or more parameters associated with each micronutrient of the one or more micronutrients based at least on the user information and artificial intelligence (AI) model,
wherein the one or more parameters associated with each of the one or more micronutrients for manufacturing the supplement for the user comprises at least an optimum dosage value, an optimal serving form and an optimal serving frequency of the supplement,
compute the time schedule to manufacture and deliver the supplement requested by the user by monitoring the digital inventory of the user and the user is notified of the time schedule or expected time of arrival of the supplement in the health application upon receiving the request to manufacture the supplement through the health application from the user;
facilitate manufacturing and delivery of the supplement_{—} with the determined one or more micronutrients according to the recommendations of the AI model for the user in a timely manner based at least on monitoring a digital inventory associated with the user in the health application; and
determining, by the server system, the authenticity of the delivered supplement based on a unique code of the supplement, wherein the unique code of the supplement is scanned and transmitted using the user device to the server system through the health application.

12. The server system as claimed in claim 11, wherein the user information is extracted on a periodic basis from the plurality of sources and the external databases for manufacturing the supplement, and wherein the user information comprises data related to a plurality of supplements comprised in the digital inventory, health metrics, dosage values, serving form, and quantity of each micronutrient available in the user's body.

13. The server system as claimed in claim 11, wherein the plurality of sources comprises a user profile of the user in the health application, the user device, and one or more wearable devices and nutrient trackers associated with the user communicably coupled to the health application, and wherein the external databases comprise one or more laboratory test reports associated with the user.

14. The server system as claimed in claim 11, wherein the server system is further caused, at least in part, to:
determine a time schedule to manufacture and deliver the supplement for the user based, at least in part, on the digital inventory associated with the user; and
transmit supplement data comprising the one or more micronutrients of the supplement and the one or more parameters associated with each micronutrient, and the time schedule to a manufacturing application available on a user device associated with a supplement manufacturer.

15. The server system as claimed in claim 11, wherein the server system is further caused, at least in part, to:
perform authentication of the supplement comprising the one or more micronutrients manufactured for the user based, at least in part, on a manufacturing supply chain network.

16. The server system as claimed in claim 11, wherein the server system is further caused, at least in part, to:
receive a feedback response from the user through the health application following the consumption of the supplement manufactured for the user for achieving a nutrition goal determined for the user in the health application;
determine efficacy of the supplement manufactured for the user based, at least in part, on the feedback response; and
provide recommendations to optimize subsequent manufacturing of the supplement for the user, if the efficacy of the supplement on the user is less than a predefined value, wherein the recommendations for optimizing subsequent manufacturing of the supplement comprises information related to optimum dosage values and serving form to be selected for each micronutrient of the supplement.

17. The server system as claimed in claim 11, wherein the server system is further caused, at least in part, to:
provide reward points in form of digital currency to the user based, at least in part, on consumption habits of the user, purchasing supplements in the health application, and user activities in the health application, wherein the reward points in the form of digital currency provided to the user are crypto collectibles.

18. The server system as claimed in claim 11, wherein the server system is further caused, at least in part, to:
render a set of features in the health application for establishing a connection between an extended reality (XR) device associated with the user and the health application available on the user device; and
facilitate display of a comparison result of two or more supplements on the XR device of the user, wherein the comparison result provides a comparison of the two or more supplements on an ingredient level comprising a list of micronutrients associated with each of the two or more supplements, thereby allowing the user to purchase a suitable supplement from the two or more supplements available in the health application.

19. The server system as claimed in claim 18, wherein the server system is further caused, at least in part, to:
determine at least one micronutrient from the list of micronutrients is incompatible for the user based, at least in part, on the user information extracted from the plurality of sources and the external databases on a periodic basis; and
upon determining the at least one micronutrient is incompatible for the user, facilitate display of an alert message in form of an indicator corresponding to the at least one micronutrient in the health application.

20. The server system as claimed in claim 11, wherein the server system is further caused, at least in part, to:
render a list of customizable settings in the health application for allowing the user to customize the digital inventory of the user on an ingredient level in the health application.
